(19) United States
(12) Patent Application Publication
(10) Pub. No.: US 2016/0038701 A1
(43) Pub. Date: Feb. 11, 2016

(54) WASH PORT ASSEMBLIES FOR AIRWAY ADAPTERS

(71) Applicant: CareFusion 2200, Inc., San Diego, CA (US)

(72) Inventors: Dennis WHITE, Yorba Linda, CA (US); Haojun FU, Yorba Linda, CA (US)

(21) Appl. No.: 14/821,447

(22) Filed: Aug. 7, 2015

Related U.S. Application Data
(60) Provisional application No. 62/035,381, filed on Aug. 8, 2014.

(51) Int. Cl. A61M 16/04; A61M 16/08; A61M 16/20

(52) U.S. Cl. CPC A61M 16/0463; A61M 16/20; A61M 16/0816; A61M 2209/10

(57) ABSTRACT

Wash port assemblies for airway adapters and methods are described. An example wash port valve assembly may include a multi-part housing defining a cavity and comprising a cap portion having a first hardness, and a base portion having a second hardness that has a hardness value greater than the first hardness. The cap portion may define an inlet port and the base portion may define an outlet port. The valve assembly may include an elastomeric valve disposed within the cavity and may be configured to be movably retained within the multi-part housing. The elastomeric valve may comprise a valve tip portion and a valve end portion distal of the valve tip portion. An example airway adapter assembly may include an adapter housing defining a cleaning chamber, and a wash port coupling assembly coupled to the wash port of the cleaning chamber.

### CROSS-REFERENCE TO RELATED APPLICATIONS

The present application claims priority to U.S. Provisional Patent Application No. 62/035,381, filed on Aug. 8, 2014, entitled, “WASH PORT ASSEMBLIES FOR AIRWAY ADAPTERS,” the disclosure of which is incorporated herein by reference in its entirety.

### TECHNICAL FIELD

The present disclosure generally relates to airway adapters and related suction catheter systems, and more particularly to wash port assemblies for airway adapters.

### BACKGROUND

Ventilators and related breathing circuits may be used to assist in patient breathing. For example, during surgery and other medical procedures, a patient may be connected to a ventilator to provide respiratory gases to the patient. The ventilation source may be connected into the patient's respiratory tract via an artificial airway, such as a tracheostomy tube, endotracheal tube, etc. While some breathing circuits can establish a single, direct fluid connection between the ventilator and the artificial airway, in many instances, caregivers desire the ability to introduce instruments and/or materials into the breathing circuit, for example, to insert instruments for visualization or related procedures, or to aspirate fluid or secretions from the patient's airway. Such instrument may require cleaning after being retrieved from the patient's airway.

For example, closed suction catheters can remain attached to a patient's airway over several days, during which suctioning procedures may be frequently repeated. In between suctioning procedures, any residual secretions or materials should be removed from the catheter surfaces. However, at least some conventional suction catheter or instrument washing procedures have proven at times to be neither safe nor effective.

### SUMMARY

Aspects of the subject technology relate to wash ports assemblies for airway adapters and methods of using the same. In accordance with certain aspects, a wash port valve assembly may comprise a multi-part housing defining a cavity and comprising a cap portion having a first hardness, and a base portion having a second hardness that has a hardness value greater than the first hardness, wherein the cap portion defines an inlet port and the base portion defines an outlet port; and an elastomeric valve disposed within the cavity and configured to be movably retained within the multi-part housing, the elastomeric valve comprising a valve tip portion and a valve end portion distal of the valve tip portion, wherein a portion of the multi-part housing is configured to secure the valve end portion of the elastomeric valve.

In accordance with certain aspects, an airway adapter assembly may comprise an adapter housing defining a cleaning chamber, the cleaning chamber comprising an access port for receiving a medical implement and a wash port; and a wash port coupling assembly coupled to the wash port of the cleaning chamber. The wash port coupling assembly may comprise a valve connector assembly comprising a valve housing defining a cavity and an elastomeric valve disposed within the cavity and configured to be movably retained within the valve housing, wherein the valve housing defines an inlet connection port and a valve outlet port with a controllable fluid passage therebetween based on a position of the elastomeric valve within the cavity, and a tubular section having a first end coupled the valve outlet port and a second end.

In accordance with certain aspects, a method for cleaning a catheter in a suction catheter system may comprise positioning a tip end of a suction catheter within a cleaning zone of an airway adapter; coupling a dispenser tip of a solution dispenser to an inlet port of a wash port valve assembly comprising a housing and an elastomeric valve, the wash port valve assembly being controllably fluidly coupled to the cleaning zone of the airway adapter; and causing solution from the solution dispenser to the flow into cleaning zone.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A illustrates a perspective view of an example of a wash port valve assembly, in accordance with aspects of the present disclosure.

FIG. 1B illustrates a cross-sectional view of the example wash port valve assembly of FIG. 1A, in accordance with aspects of the present disclosure.

FIG. 1C illustrates an enlarged cross-sectional detail view of the example wash port valve assembly of FIG. 1A, in accordance with aspects of the present disclosure.

FIG. 2A illustrates a perspective view of another example of a wash port valve assembly, in accordance with aspects of the present disclosure.

FIG. 2B illustrates a cross-sectional view of the example wash port valve assembly of FIG. 2A, in accordance with aspects of the present disclosure.

FIGS. 2C-2E illustrate cross-sectional views of another example wash port valve assembly, in accordance with aspects of the present disclosure.

FIGS. 3A and 3B illustrate perspective views of an example of a multiple-port airway access adapter with a wash port coupling assembly, in accordance with aspects of the present disclosure.

FIG. 4 illustrates a cross-sectional perspective view of an example of a multiple-port airway access adapter with a wash port coupling assembly in use with a suction catheter, in accordance with aspects of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components may be shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

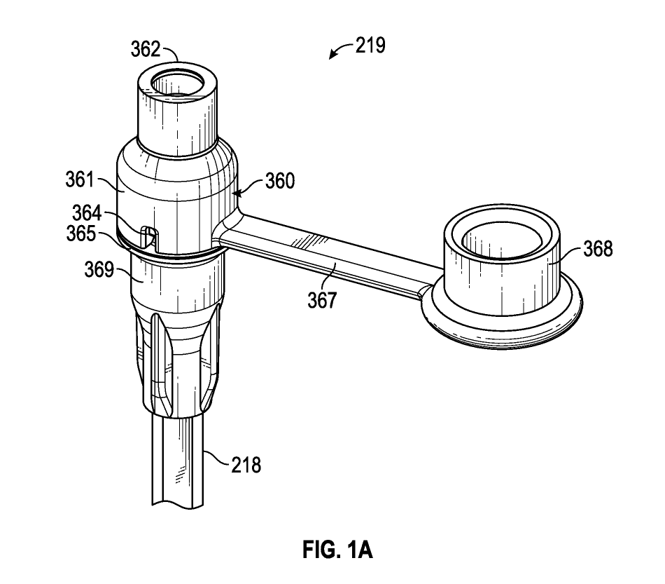

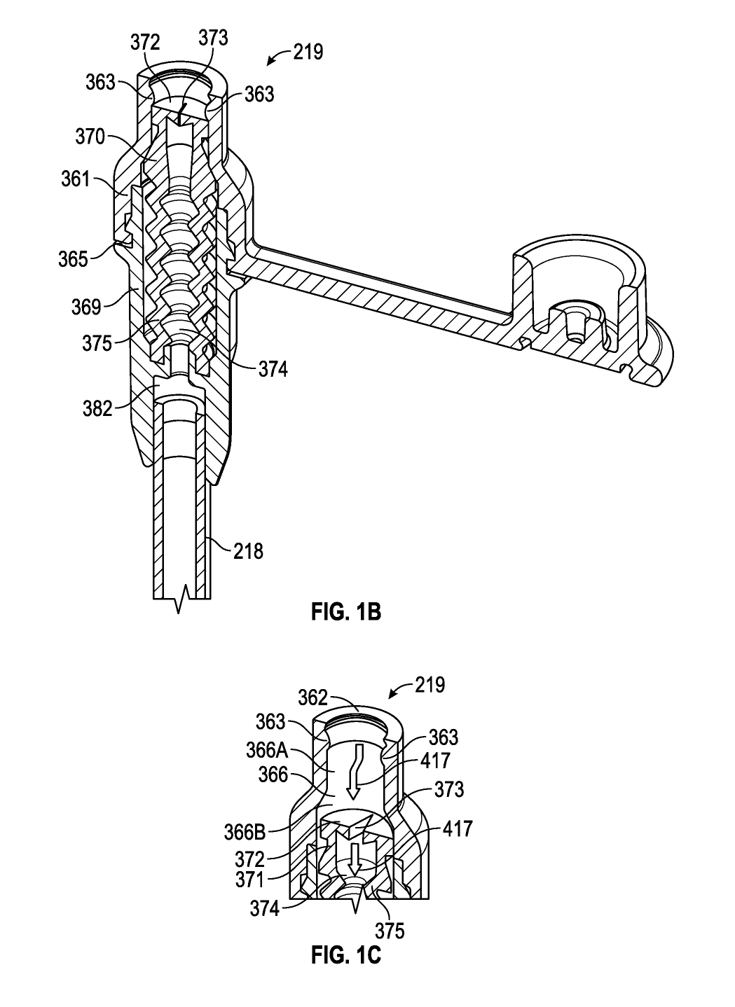

FIGS. 1A-1C illustrates an example of a wash port valve assembly 219. In certain embodiments, wash port valve assembly 219 comprises a multi-part valve housing 360 and an elastomeric valve 370 disposed within the multi-part valve housing 360. Multi-part valve housing 360 may be formed from a valve body cap portion 361 and a valve body base portion 369. In accordance with certain embodiments, each of the valve body cap portion 361, valve body base portion 369, and elastomeric valve 370 may be formed from different material and/or similar materials having different characteristics and properties. In some embodiments, one or both of the valve body cap portion 361 and valve body base portion 369 may be substantially rigid and generally cylindrically shaped. In other embodiments, however, a single housing and/or single material may be used.

For example, valve body cap portion 361 may be formed from an elastomeric material such as, but not limited to, a thermoplastic elastomer. In certain embodiments, the elastomeric material of the valve body cap portion 361, and an inlet port 362 thereof, may have a durometer value between approximately 83 +/−5 and 90 +/−5 on a Shore A hardness scale. In this regard, the elastomeric material may be pliable so that when a dispense tip (e.g., a male luer-tapered tip) of a solution dispenser (e.g., a saline bullet or a syringe) is engaged with the opening of the inlet port 362 of the valve body cap portion 361, the solution dispenser can remain securely in its place without a user or caregiver holding the solution dispenser, as well as when the wash port valve assembly 219 moves or swings around so as to contact a patient or other object, for example, while a cleaning operation is being performed.

Additionally, in some embodiments, the inlet port 362 may include an elongate channel portion having a retaining ridge 363 disposed on an interior surface of the elongate channel portion. When receiving a dispense tip of a solution dispenser, the retaining ridge 363 may expand and flatten along with the inlet port 362 so as to securely grip the dispense tip. Accordingly, valve body cap portion 361 may provide for better connection with and retention of a solution dispenser (or other medical implement). It is to be appreciated that a failure mode while performing a cleaning operation (e.g., washing a closed suction catheter in a multi-port airway adapter), which may involve a user or caregiver attempting to attach either a saline bullet or a syringe into a wash portal, only to have the bullet or syringe prematurely disconnect, can be eliminated or substantially avoided by utilizing wash port valve assemblies disclosed herein.

A housing seal 365 of the multi-part valve housing 360 may be included to fixably couple the different materials or variations of the materials used to form the valve body cap portion 361 and the valve body base portion 369, for example. In certain embodiments, a seal access port 364 may be arranged such that the valve body cap portion 361 and the valve body base portion 369 may be bonded using adhesives. In some implementations, the multi-part valve housing 360 may be assembled by securing the elastomeric valve 370 to the valve body base portion 369. For example, an end distal of valve tip 371 of the elastomeric valve 370 may be coupled or secured to a section of valve body base portion 369 proximate to valve outlet port 382. The valve body cap portion 361 may then be latched onto the valve body base portion 369 and bonded together using adhesives injected into housing seal 365 via seal access port 364.

In accordance with certain embodiments, elastomeric valve 370 may be axially compressible and/or collapsible when arranged in the multi-part valve housing 360. Additionally, tubular connector 218 can comprise a flexible tube segment and may be coupled to the valve outlet port 382, for example, by bonding, ultrasonic welding, or interference fitting, compression coupling, or the like. However, it is to be understood that other processes of assembling are contemplated, in accordance with various implementations and embodiments of the present disclosure. Moreover, it is to be understood that wash port valve assembly 219 and tubular connector 218 may be configured for permanent or semi-permanent attachment together and/or to a suction catheter system.

In certain embodiments, valve body cap portion 361 may include a valve cap cover 368 coupled to the valve body cap portion 361 by a tether 367. The valve body cap portion 361, tether 367, and valve cap cover 368 may be integrally formed in accordance with some embodiments.

Valve body base portion 369 and valve outlet port 382 of the valve body base portion 369 may be formed of a material harder than the material of valve body cap portion 361. For example, valve body base portion 369 may be formed of polycarbonate having a durometer value of approximately 80 +/−5 on a Shore D hardness scale. In some embodiments, however, the polycarbonate of valve body base portion 369 may have a hardness value ranging from 50 to 121 per ISO 2039-1. In other embodiments, body base portion 369 may be formed of a material other than polycarbonate.

Moreover, elastomeric valve 370 may be formed of a material softer than the material of valve body cap portion 361. For example, in some implementations, all or some of elastomeric valve 370 may be comprised of liquid silicone rubber elastomeric material such as commercially available silicone having durometer value in the range approximately 30 to 80 on a Shore A hardness scale. Other implementations, however, may be formed from a harder elastomeric material, for example, when an elastomeric valve is used to form a seal with a housing portion of a valve assembly.

In certain embodiments, multi-part valve housing 360 may form a cavity 366 in which at least a portion of the elastomeric valve 370 is movable. For example, cavity 366 may comprise a narrower portion 366a and a wider portion 366b in which elastomeric valve 370 may operate and be retained. In certain embodiments, when elastomeric valve 370 is unbiased within multi-part valve housing 360 (e.g., the elastomeric valve 370 is not in contact with a dispensing tip), valve tip 371 and valve tip face 372 may be positioned within the elongate channel portion a distance from the opening of inlet port 362. Moreover, the retaining ridge 363 may comprise an inwardly-extending circumferential ridge or ring structure disposed within the interior surface of the elongate channel portion between the opening of the inlet port 362 and the elastomeric valve 370 in an unbiased state. However, in other configurations and embodiments, valve tip face 372 may be positioned within the elongate channel portion approximately flush with the opening of inlet port 362 in an unbiased state.

In certain embodiments, a valve tip 371 comprises one or more slits 373 that can be forced closed by the narrower cavity portion 366a of the elongate channel portion of the inlet port 362 (FIGS. 1B and 1C). In this regard, the one or more slits 373 are normally closed and fluid-tight when inlet port 362 of wash port valve assembly 219 is not engaged with or connected to a dispenser tip of a solution dispenser. In other configurations, slits of the valve tip may include an “X” or “*” shaped cross-cuts, for example.

In operation, the elastomeric valve 370 may be caused to in a biased state within the multi-part valve housing 360. For example, when an axial force is applied against the valve tip face 372, the elastomeric valve 370 may axially compress such that the valve tip 371 extends into the wider cavity portion 366b of the multi-part valve housing 360. Accordingly, the valve tip 371 may automatically open such that fluid passage 417 through the valve tip 371 and into an internal passage 374 of the elastomeric valve 370 can occur (FIG. 1C). The internal passage 374 may be fluidly coupled with valve outlet port 382 to further fluid passage 417 to the tubular connector 218, for example.

In certain embodiments, one or more accordion bellows 375 may be disposed on a wall section of the elastomeric valve 370 that defines the internal passage 374. When in the biased state, the one or more accordion bellows 375 may become compressed such that the elastomeric valve 370 can spring back to the unbiased state when the force applied to the valve tip face 372 is removed. As such, the valve tip 371 may be guided back into the narrower cavity portion 366a of the multi-part valve housing 360 as the elastomeric valve 370 springs back or decompresses so that the one or more slits 373 in the valve tip 371 close and occlude fluid flow through the wash port valve assembly 219.

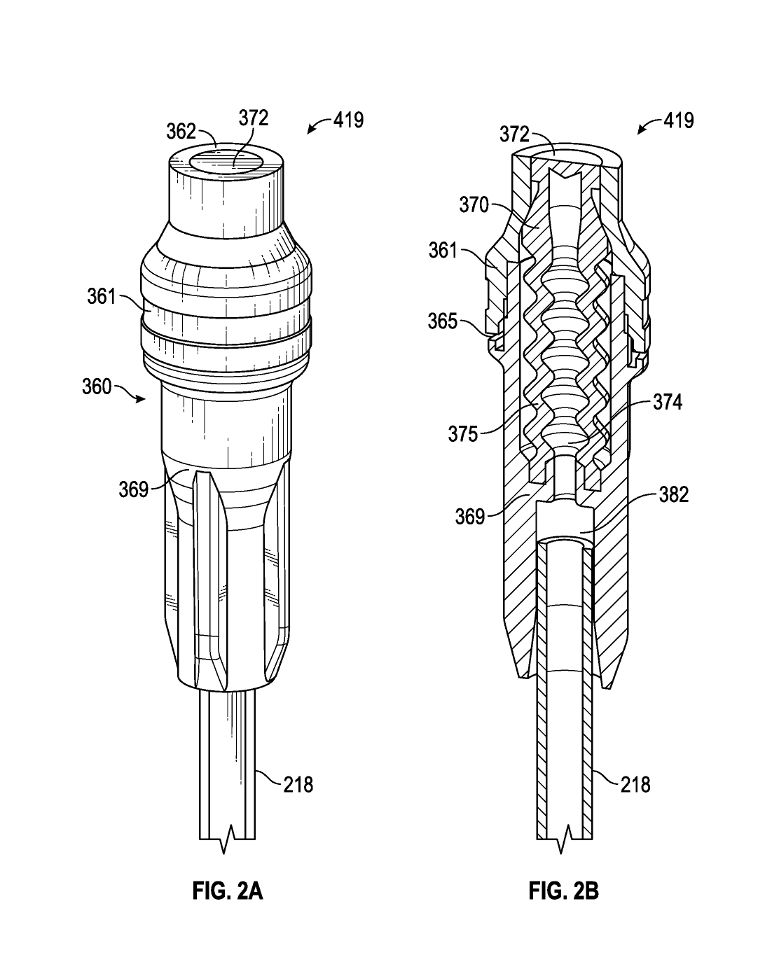

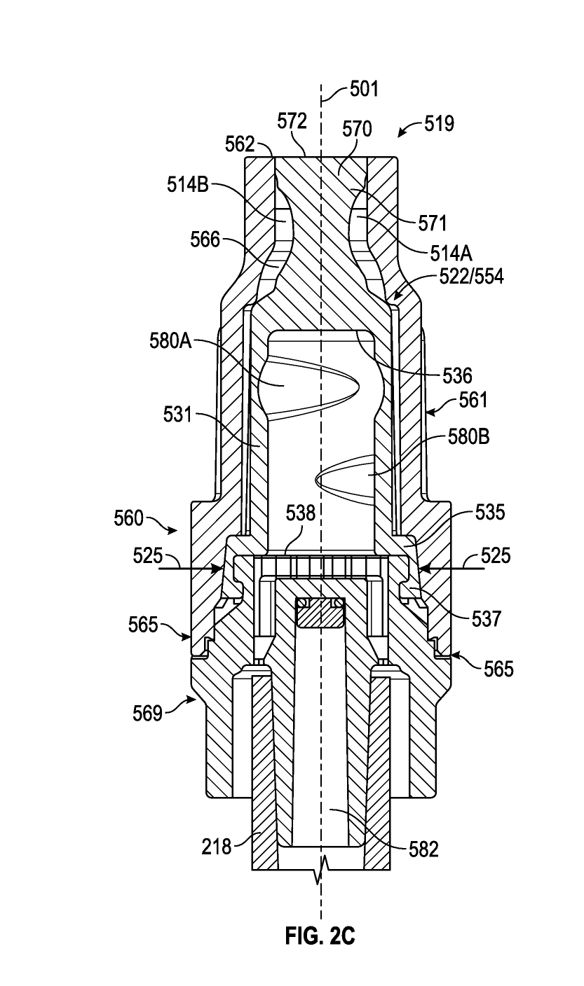

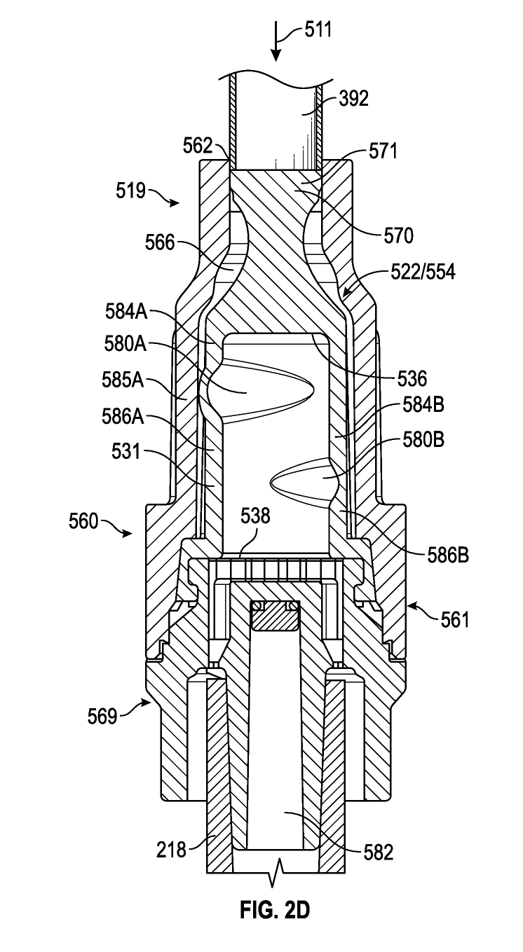

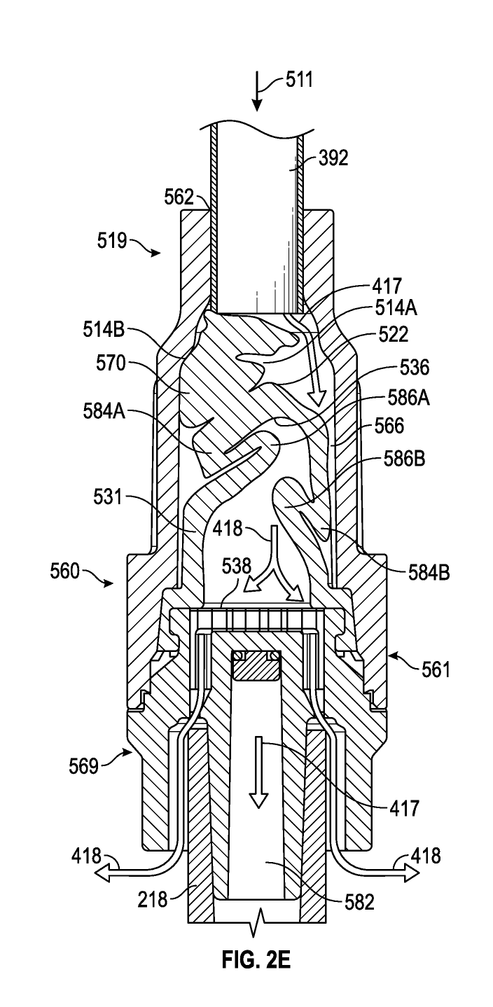

FIGS. 2A and 2B illustrate another example of a wash port valve assembly 419. In some embodiments, wash port valve assembly 419 may include similar features, geometries, and components as wash port valve assembly 219. However, elastomeric valve 370 may be positioned in multi-part valve housing 360 such that valve tip face 372 is substantially flush with the opening of the inlet port 362 of wash port valve assembly 419. Additionally, multi-part valve housing 360 of wash port valve assembly 419 may be longer than wash port valve assembly 219, in accordance with some configurations. Moreover, in some embodiments, valve body cap portion 361 may be ultrasonically welded with valve body base portion 369 to form the multi-part valve housing 360 of wash port valve assembly 419.

It is to be understood that many configurations and embodiments of wash port valve assemblies are contemplated in the present disclosure, including, for example, valve assemblies configured for negative, neutral, and positive fluid displacement. Wash port valve assembly embodiments 219, 419 may be configured as negative to neutral fluid displacement connection types. Additionally, wash port valve assembly embodiments 219, 419, may be configured for quick and secure closure upon removal of a dispenser tip from the inlet port 362 of such embodiments. Accordingly, if fluid pressure has increased inside the cleaning chamber of airway adapter 100, when the dispenser tip 392 of the solution dispenser 390 is withdrawn from the inlet port 562, closed positioning of the elastomeric valve 370 may prevent a pressurized fluid from spraying out towards the user or caregiver performing a cleaning operation of a suction catheter, for example.

FIGS. 2C-2E illustrate another example of a wash port valve assembly 519 that may be configured for positive to neutral fluid displacement. FIG. 2C provides a longitudinal cross-sectional view of wash port valve assembly 519 showing the compressible elastomeric valve 570 in a multi-part valve housing 560 formed by valve body cap portion 561 and valve body base portion 569. The cross-sectional view of FIG. 2C is of exemplary wash port valve assembly 519 along cross section of a wash port valve assembly similar to that of the wash port valve assembly 419 (with differences in the multi-part valve housing design) shown in FIG. 2A. The assembled wash port valve assembly 519 as illustrated in FIG. 2C is in a sealed configuration such that any fluid from an interconnected fluid passage or path coupled to the valve outlet port 582 (e.g., a cleaning chamber of airway adapter 100) is sealed from the inlet port 562.

Wash port valve assembly 519 may be assembled such that a flange portion 535 of the compressible elastomeric valve 570 is coupled or snapped onto a valve mount of the valve body base portion 569. For example, the flange portion 535 may include an inwardly facing lip 537 and partial transverse upper wall abutting the elongate cylindrical wall 531 such that the flange portion 535 can securely engage with the valve mount. The rim of the valve mount may include an upper partial transverse wall and a lower partial transverse wall to facilitate such a secure engagement with the flange portion 535 and compressible elastomeric valve 570.

An opening of the valve body cap portion 561 may be arranged such that a valve head 571 (or valve tip portion) of the compressible elastomeric valve 570 is aligned and disposed within a port channel of the inlet port 562. Upon assembly, the top surface 572 of the valve head 571 of the compressible elastomeric valve 570 may have a resulting plane that is substantially perpendicular to the central longitudinal axis 501 or axial center of a column section of the valve head 571 when the valve head 571 is engaged within the port channel of the valve body cap portion 561 of the multi-part valve housing 560. However, in some embodiments, the top surface 572 of the valve head 571 may be countersunk within the port channel of the valve body cap portion 561 similar to the wash port valve assembly 219 embodiments illustrated in FIGS. 1A and 1B and described herein.

Additionally, one or more internal contact tabs may be disposed on the lower section of the valve body cap portion 561 that surround and apply pressure to a sidewall of the flange portion 535 to secure or anchor the compressible elastomeric valve 570 in the multi-part valve housing 560. In operation, the compressible elastomeric valve 570 of the wash port valve assembly 519 can compress and collapse when an axial force is applied to the top surface 572 of the compressible elastomeric valve 570 and expand and realign when the axial force is removed. In some implementations, all or some of elastomeric valve 370 may have a durometer value of approximately 70 +/−5 on a Shore A hardness scale.

Accordingly, the one or more internal contact tabs can provide a radial force 525 substantially orthogonal to the central longitudinal axis 501 onto the sidewall of the flange portion 535 and the rim of the valve body base portion 569. In this regard, when an axial force is applied to the top surface 572 of the valve head 571 of the compressible elastomeric valve 570, the effect of any resulting axial force through the compressible elastomeric valve 570 onto the valve body base portion 569 is reduced if not eliminated. Such a resulting axial force applied onto the valve body base portion 569 can work against or in derogation to the housing seal/fused connection 565 of the valve body base portion 569 and the valve body cap portion 561, and over time may cause the fused connection 565 to become breached and/or separated.

Valve body cap portion 561 may include an internal cavity 566 and an internal sealing edge 554. The internal sealing edge 554 may be a circumferential edge and configured for retaining a compressible valve within the internal cavity 566 of an assembled wash port valve assembly 519. In operation, the internal sealing edge 554 may be arranged to provide blocking of fluid flow in conjunction with a surface of primary seal portion 522 of the compressible elastomeric valve 570.

Still referring to FIGS. 2C-2E, a lower portion of the compressible elastomeric valve 570 may include a closed end 536 proximal to the primary seal portion 522 and an open end 538 distally of the primary seal portion 522. An interior wall of the lower portion may define, in part, an interior air space of the compressible elastomeric valve 570. The lower portion of the compressible elastomeric valve 570 may include various interior dimples, incisions, discontinuity segments, or the like disposed along the interior wall to facilitate proper compressing and collapsing functionality for operation of zero reflux and/or positive displacement aspects associated with various wash port valve assembly 519 embodiments.

For example, a first interior dimple 580a and a second interior dimple 580b may be disposed on and along the interior wall of the compressible elastomeric valve 570. The first interior dimple 580a and the second interior dimple 580b may be disposed on opposite sides of the interior wall and at longitudinally different positions. Moreover, the size and shape of each interior dimple 580a, 580b may be distinct. In certain embodiments, the first interior dimple 580a may be larger than the second interior dimple 580b. Moreover, a first notch 514a of the valve head 571 may be longitudinally aligned with respect to the second internal dimple 580b, and a second notch 514b may be longitudinally aligned with respect to the first interior dimple 580a.

The first interior dimple 580a may be disposed proximal to the primary seal portion 522, and the second interior dimple 580b may be disposed distally of the first interior dimple 580a. Both the first and second interior dimples 580a, 580b may be disposed along the interior wall of the elongate cylindrical wall 531 with respect to the central longitudinal axis 501.

Referring to FIG. 2D, the compressible elastomeric valve 570 of the wash port valve assembly 519 is shown upon initial entry of a dispenser tip 392 of a solution dispenser 390 (or other medical implement such as, but not limited to, a syringe) into the inlet port 562. The cross-sectional view of FIG. 2D is of the wash port valve assembly 519 as would be modified by the described valve operation.

As the dispenser tip 392 is initially inserted into the inlet port 562 of the wash port valve assembly 519, an initial axial force 511 is exerted onto the compressible elastomeric valve 570 such that a central portion 585a of the elongate cylindrical wall 531 proximal to the first interior dimple 580a may slightly bow out toward the inner wall of the valve body cap portion 561. Additionally, the exterior surface (e.g., frustoconical section) of the primary seal portion 522 may separate from the internal sealing edge 554.

In the example of FIG. 2E, a view of wash port valve assembly 519 is provided showing the compressible elastomeric valve 570 after further entry of the dispenser tip 392 into the inlet port 562. The cross-sectional view of FIG. 2D is of the wash port valve assembly 519 as would be modified by the described valve operation.

Upon additional axial force 511, the dispenser tip 392 may descend further into the inlet port 562, further compressing, collapsing, canting, and/or folding may occur with respect to certain sections of the compressible elastomeric valve 570. For example, as illustrated in FIG. 2E, a bottom portion 586a of the elongate cylindrical wall 531 proximal to the first interior dimple 580a may fold inwardly and underneath a top portion 584a. Similarly, a bottom portion 586b of the elongate cylindrical wall 531 proximal to the second interior dimple 580b may fold inwardly and underneath a top portion 584b.

Additionally, the first notch 514a may fold or collapse and the second notch 514b may open or expand such that a first top edge portion of the top surface 572 disposed generally above the first notch 514a of the valve head 571 may tilt downwardly. In this regard, fluid passage 417 from the dispenser tip 392 in the inlet port 562 may be established through the interior of the wash port valve assembly 519 to the valve outlet port 582. For example, a fluid passage 417 may be established between the inlet port 562 and the valve outlet port 582 via the internal cavity 566 around the exterior of the compressible elastomeric valve 570 and through fluid flow channels defined within interior support columns (not shown in the cross-section view) on the interior of the valve body cap portion 561. Fluid passage 417 may extend from the fluid flow channels of the valve body cap portion 561 to the valve body base portion 569 and into the valve outlet port 582.

Moreover, a second top edge portion of the top surface 572 disposed generally above the second notch 514b, the second notch 514b, and/or a portion of the surface of the primary seal portion 522 disposed proximal to the second notch 514b may be positioned such that they predominantly contact an interior support column (not shown) of the valve body cap portion 561 while in the compressed configuration.

When the dispenser tip 392 is removed from the inlet port 562, the compressible elastomeric valve 570 may return to its position within the multi-part valve housing 560 in a sealed configuration as illustrated in FIG. 2C. Thus, if fluid pressure has increased inside the cleaning chamber of airway adapter 100, when the dispenser tip 392 of the solution dispenser 390 is withdrawn from the inlet port 562, positive displacement of any fluid in the wash port valve assembly 519 and/or the closed positioning of the compressible elastomeric valve 570 may prevent a pressurized fluid from spraying out towards the user or caregiver performing a cleaning operation of a suction catheter, for example.

Wash port valve assembly 519 illustrated in the example of FIGS. 2C-2E may include additional aspects and features such as, but not limited to, those disclosed in U.S. patent application Ser. No. 14/149,753, filed Jan. 7, 2014, the entire disclosure of which is hereby incorporated by reference.

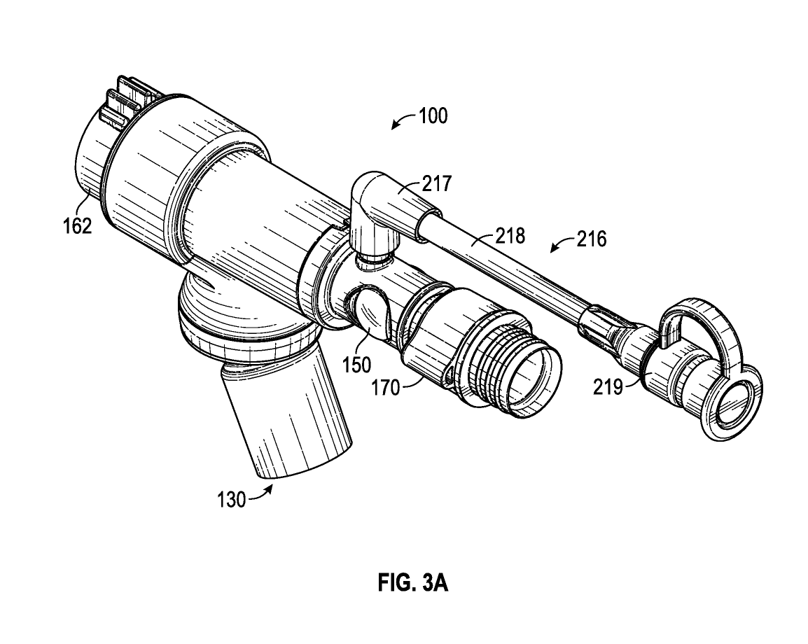

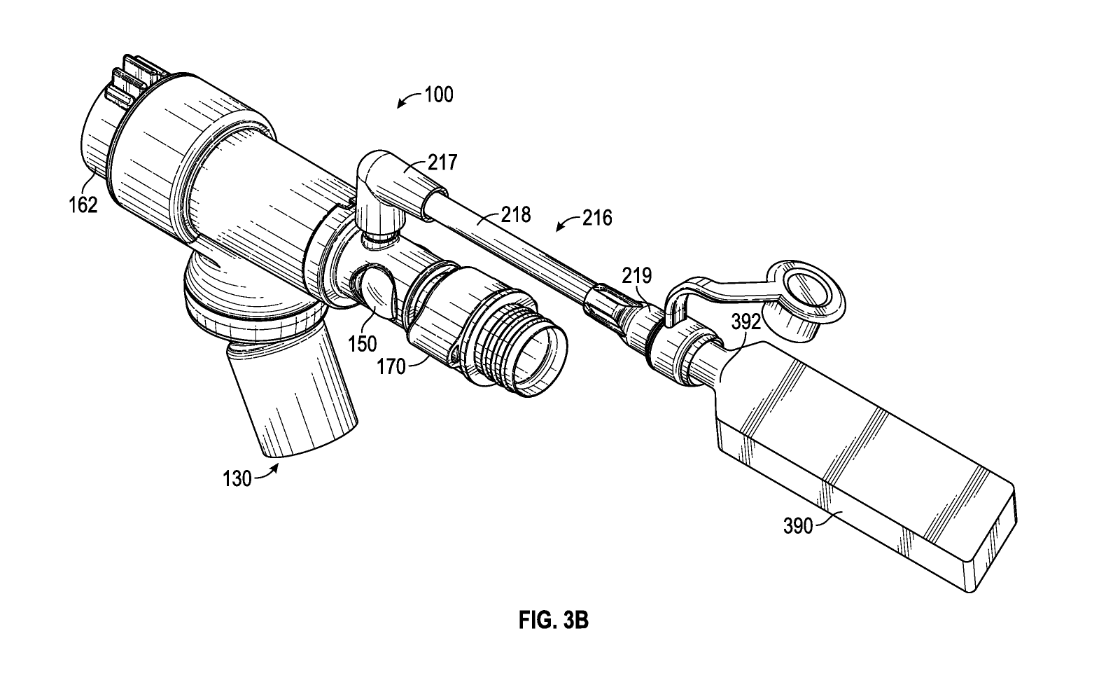

Referring to the example of FIG. 3A, a multiple-port airway access adapter may include a wash port coupling assembly. Airway adapter 100 may include an airway adapter coupler 170, lens 150, ventilator port 130, swivel connector 162, and a wash port coupling assembly 216. Wash port coupling assembly 216 may comprise wash port valve assembly 219, tubular connector 218, and elbow connector 217.

As illustrated in the example of the FIG. 3B, a solution dispenser 390 may be coupled to the wash port coupling assembly 216 by inserting a dispensing tip 392 of the solution dispenser 390 into the wash port valve assembly 219 (or valve connector assembly).

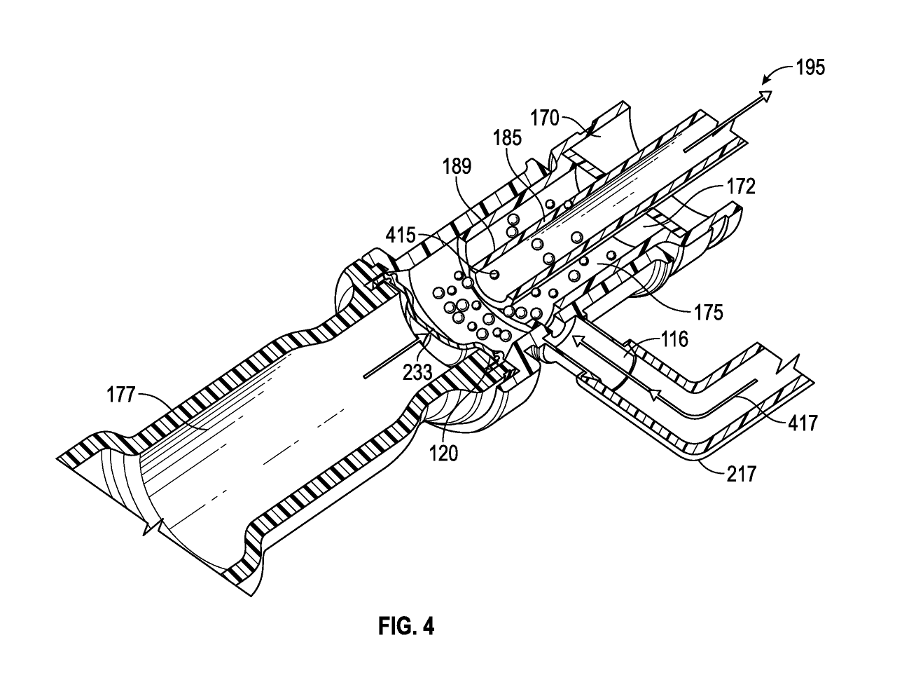

FIG. 4 illustrates an example of an airway access adapter with a wash port coupling assembly in use with a suction catheter. Referring to the example in FIG. 4, an example cleaning process associated with an airway adapter is described. It is to be understood that the operations described herein may be used in conjunction with other processes and aspects of the present disclosure. Although aspects of example cleaning process may be described with relation to the examples provided in FIG. 4, as well as the other figures in the present disclosure, the example processes and operations are not limited to such.

For example, a tip end 189 of a suction catheter 185 may be positioned within a cleaning zone 175 (e.g., a cleaning chamber or access zone) of the airway adapter. The cleaning zone 175 of the airway adapter may be fluidly isolated from a ventilation zone 177 of the airway adapter by a valve 120 configured to create a controllable fluid barrier. The cleaning zone 175 may be used to remove substances such as mucus, secretions, and other fluids from the lungs that may have accumulated on the suction catheter 185.

In some embodiments, the suction catheter 185 may be aligned or positioned such that the tip end 189 of the suction catheter 185 is positioned between valve 120 and an airway adapter coupler 170. For example, airway adapter coupler 170 may include a wiper seal 172 or other barrier for forming an end of the cleaning zone 175.

A dispenser tip 392 of a solution dispenser 390 may be coupled to an inlet port of a wash port valve assembly 219 (FIG. 3B). Wash port valve assembly 219, 419, 519 may comprise a housing and an elastomeric valve (FIGS. 1A-1C and 2A-2E). In accordance with certain embodiments, wash port valve assembly may be controllably fluidly coupled to the cleaning zone 175 of the airway adapter. For example, wash port valve assembly may be fluidly coupled to an elbow connector 217. The elbow connector 217 may be coupled to a wash port 116 (or flush port) that provides fluid access to the cleaning zone 175 of the airway adapter.

A solution (e.g., saline or a cleaning solution) contained in the solution dispenser may be caused to flow from the solution dispenser into the cleaning zone 175. For example, the solution may be injected or squeezed into the wash port valve assembly. Alternatively, or additionally, the solution may be caused to flow when the dispenser tip engages the elastomeric valve such that fluid passage is allowable through the wash port valve assembly configured for controllable fluid communication, and a suction force 195 is applied to the suction catheter 185.

Additionally, in some embodiments, the suction force 195 may be applied to the suction catheter 185 such that the secondary seal 233 of the valve 120 is breached, causing airflow from the ventilation zone 177 into the access zone 175. In this regard, an airway adapter comprising valve 120 may include a volume (e.g., cleaning zone 175) for cleaning a catheter tip or other medical implement (e.g., Mini-BAL device or a bronchoscope) following a suctioning or other airway procedure. Valve 120 may be configured to allow a small amount of air (relative to the amount and flow generated by a ventilation source of a particular patient's artificial airway circuit) through the seal so as to entrain air into the fluid flow of solution from the solution dispenser thereby improving the cleaning procedure effectiveness. In this regard, air entrainment traces 415, in addition to solution traces 417 are illustrated in the example of FIG. 4, in accordance with certain embodiments of the present disclosure.

Moreover, the solution dispenser may begin to collapses from fluid being suctioned therefrom in a non-vented manner (e.g., from a saline bullet). Thus, as noted herein, features associated the inlet port and connection to the wash port valve assembly may aid in keeping the dispenser tip securely coupled within the inlet port through completion of the cleaning procedure.

The dispenser tip of the solution dispenser may be decoupled from the inlet port of the wash port valve assembly. As also noted herein, when the dispenser tip of the solution dispenser is withdrawn from the inlet port, the wash port valve assembly may be configured to return to a closed positioning of the elastomeric valve 370 and/or provide positive fluid displacement to prevent reduce the possibility of pressurized fluid within the cleaning zone 175 from spraying out towards the user or caregiver performing a cleaning operation. In this regard, when the dispenser tip is decoupled, the wash port valve assembly may be configured to occlude fluid passage from the cleaning zone 175 of the airway adapter to an opening of the inlet port of the wash port valve assembly.

The subject technology is illustrated, for example, according to various aspects described above. Various examples of these aspects are described as numbered concepts or clauses (1, 2, 3, etc.) for convenience. These concepts or clauses are provided as examples and do not limit the subject technology. It is noted that any of the dependent concepts may be combined in any combination with each other or one or more other independent concepts, to form an independent concept. The following is a non-limiting summary of some concepts presented herein:

Concept 1. A wash port valve assembly comprising:

a multi-part housing defining a cavity and comprising a cap portion having a first hardness, and a base portion having a second hardness that has a hardness value greater than the first hardness, wherein the cap portion defines an inlet port and the base portion defines an outlet port; and

an elastomeric valve disposed within the cavity and configured to be movably retained within the multi-part housing, the elastomeric valve comprising a valve tip portion and a valve end portion distal of the valve tip portion,

wherein a portion of the multi-part housing is configured to secure the valve end portion of the elastomeric valve.

Concept 2. The wash port valve assembly of concept 1 or any other concept, wherein the elastomeric valve has a third hardness that has a hardness value less than the first harness.

Concept 3. The wash port valve assembly of concept 1 or any other concept, wherein the first harness has a durometer value between approximately 83 +/−5 and 90 +/−5 on a Shore A hardness scale.

Concept 4. The wash port valve assembly of concept 1 or any other concept, wherein the cavity comprises a narrower cavity portion proximate to the inlet port and a wider cavity portion distal of the inlet port.

Concept 5. The wash port valve assembly of concept 4 or any other concept, wherein the cap portion further defines an inwardly-extending retaining ridge disposed on an interior surface of the narrower cavity portion proximate to an opening of the inlet port.

Concept 6. The wash port valve assembly of concept 5 or any other concept, wherein the valve tip portion of the elastomeric valve is positioned in the narrower cavity portion a distance from the opening of the inlet port and the retaining ridge when the elastomeric valve is in an unbiased state.

Concept 7. The wash port valve assembly of concept 4 or any other concept, wherein the valve tip portion of the elastomeric valve comprises a slit on a face of the valve tip portion extending through the valve tip portion to an internal passage of the elastomeric valve.

Concept 8. The wash port valve assembly of concept 7 or any other concept, wherein the slit on the face of the valve tip portion is configured to automatically open and expose the internal passage when a force is applied to the face of the valve tip portion such that the valve tip portion is positioned within the wider cavity portion.

Concept 9. The wash port valve assembly of concept 7 or any other concept, wherein the elastomeric valve comprises a circumferential wall section having one or more accordion bellows and defining at least in part the internal passage.

Concept 10. The wash port valve assembly of concept 7 or any other concept, wherein the internal passage of the elastomeric valve is fluidly coupled to the outlet port of the base portion.

Concept 11. The wash port valve assembly of concept 3 or any other concept, wherein a fluid passage is defined from the inlet port around an exterior of the elastomeric valve to the outlet port of the base portion when a force is applied to a face of the valve tip portion such that the elastomeric valve is compressed at least partially and at least a portion of the face of the valve tip portion is positioned within the wider cavity portion.

Concept 12. The wash port valve assembly of concept 1 or any other concept, wherein the cap portion comprises a tethered valve cap cover.

Concept 13. The wash port valve assembly of concept 1 or any other concept, wherein the multi-part housing comprises a seal access port proximate to a housing seal between the cap portion and the base portion.

Concept 14. An airway adapter assembly comprising:

an adapter housing defining a cleaning chamber, the cleaning chamber comprising an access port for receiving a medical implement and a wash port; and

a wash port coupling assembly coupled to the wash port of the cleaning chamber and comprising:
a valve connector assembly comprising a valve housing defining a cavity and an elastomeric valve disposed within the cavity and configured to be movably retained within the valve housing, wherein the valve housing defines an inlet connection port and a valve outlet port with a controllable fluid passage therebetween based on a position of the elastomeric valve within the cavity, and

a tubular section having a first end coupled the valve outlet port and a second end.

Concept 15. The airway adapter assembly of concept 14 or any other concept, wherein the second end of the tubular section comprises an elbow connector.

Concept 16. The airway adapter assembly of concept 14 or any other concept, wherein the housing of the valve connector assembly is a multi-part housing comprising a cap portion having a first hardness, and a base portion having a second hardness that has a hardness value greater than the first hardness.

Concept 17. The airway adapter assembly of concept 14 or any other concept, wherein the elastomeric valve comprises a valve face that is substantially flush with an opening of the inlet connection port when the elastomeric valve is in an unbiased state.

Concept 18. The airway adapter assembly of concept 14 or any other concept, wherein the inlet connection port of valve connector assembly comprises an elongate channel section extending from an opening of the inlet connection port and having an interior surface for defining at least part of the cavity, the elongate channel comprising an inwardly-extending retaining ridge disposed on the interior surface proximate to the opening.

Concept 19. The airway adapter assembly of concept 18 or any other concept, wherein the elastomeric valve comprises a valve face that is countersunk within the elongate channel section relative to the opening of the inlet connection port when the elastomeric valve is in an unbiased state.

Concept 20. A method for cleaning a catheter in a suction catheter system, the method comprising:

positioning a tip end of a suction catheter within a cleaning zone of an airway adapter;

coupling a dispenser tip of a solution dispenser to an inlet port of a wash port valve assembly comprising a housing and an elastomeric valve, the wash port valve assembly being controllably fluidly coupled to the cleaning zone of the airway adapter; and

causing solution from the solution dispenser to the flow into cleaning zone.

Concept 21. The method of concept 20 or any other concept, further comprising:

decoupling the dispenser tip of the solution dispenser from the inlet port of the wash port valve assembly such that when the dispenser tip is decoupled, the wash port valve assembly is configured to occlude fluid passage from the cleaning zone of the airway adapter to an opening of the inlet port of the wash port valve assembly.

It is understood that any specific order or hierarchy of blocks in the processes disclosed is an illustration of example approaches. Based upon design or implementation preferences, it is understood that the specific order or hierarchy of blocks in the processes may be rearranged, or that all illustrated blocks may or may not be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed provide exemplary approaches. Based upon implementation specifics or preferences, it is understood that the specific order or hierarchy of steps, operations or processes may be rearranged. Some of the steps, operations or processes may be performed simultaneously and some may be omitted. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A wash port valve assembly comprising:
a multi-part housing defining a cavity and comprising a cap portion having a first hardness, and a base portion having a second hardness that has a hardness value greater than the first hardness, wherein the cap portion defines an inlet port and the base portion defines an outlet port; and
an elastomeric valve disposed within the cavity and configured to be movably retained within the multi-part housing, the elastomeric valve comprising a valve tip portion and a valve end portion distal of the valve tip portion,
wherein a portion of the multi-part housing is configured to secure the valve end portion of the elastomeric valve.

2. The wash port valve assembly of claim 1, wherein the elastomeric valve has a third hardness that has a hardness value less than the first harness.

3. The wash port valve assembly of claim 1, wherein the first harness has a durometer value between approximately 83 +/−5 and 90 +/−5 on a Shore A hardness scale.

4. The wash port valve assembly of claim 1, wherein the cavity comprises a narrower cavity portion proximate to the inlet port and a wider cavity portion distal of the inlet port.

5. The wash port valve assembly of claim 4, wherein the cap portion further defines an inwardly-extending retaining ridge disposed on an interior surface of the narrower cavity portion proximate to an opening of the inlet port.

6. The wash port valve assembly of claim 5, wherein the valve tip portion of the elastomeric valve is positioned in the narrower cavity portion a distance from the opening of the inlet port and the retaining ridge when the elastomeric valve is in an unbiased state.

7. The wash port valve assembly of claim 4, wherein the valve tip portion of the elastomeric valve comprises a slit on a face of the valve tip portion extending through the valve tip portion to an internal passage of the elastomeric valve.

8. The wash port valve assembly of claim 7, wherein the slit on the face of the valve tip portion is configured to automatically open and expose the internal passage when a force is applied to the face of the valve tip portion such that the valve tip portion is positioned within the wider cavity portion.

9. The wash port valve assembly of claim 7, wherein the elastomeric valve comprises a circumferential wall section having one or more accordion bellows and defining at least in part the internal passage.

10. The wash port valve assembly of claim 7, wherein the internal passage of the elastomeric valve is fluidly coupled to the outlet port of the base portion.

11. The wash port valve assembly of claim 3, wherein a fluid passage is defined from the inlet port around an exterior of the elastomeric valve to the outlet port of the base portion when a force is applied to a face of the valve tip portion such that the elastomeric valve is compressed at least partially and at least a portion of the face of the valve tip portion is positioned within the wider cavity portion.

12. The wash port valve assembly of claim 1, wherein the cap portion comprises a tethered valve cap cover.

13. The wash port valve assembly of claim 1, wherein the multi-part housing comprises a seal access port proximate to a housing seal between the cap portion and the base portion.

14. An airway adapter assembly comprising:
an adapter housing defining a cleaning chamber, the cleaning chamber comprising an access port for receiving a medical implement and a wash port; and
a wash port coupling assembly coupled to the wash port of the cleaning chamber and comprising:
a valve connector assembly comprising a valve housing defining a cavity and an elastomeric valve disposed within the cavity and configured to be movably retained within the valve housing, wherein the valve housing defines an inlet connection port and a valve outlet port with a controllable fluid passage therebetween based on a position of the elastomeric valve within the cavity, and
a tubular section having a first end coupled the valve outlet port and a second end.

15. The airway adapter assembly of claim 14, wherein the second end of the tubular section comprises an elbow connector.

16. The airway adapter assembly of claim 14, wherein the housing of the valve connector assembly is a multi-part housing comprising a cap portion having a first hardness, and a base portion having a second hardness that has a hardness value greater than the first hardness.

17. The airway adapter assembly of claim 14, wherein the elastomeric valve comprises a valve face that is substantially flush with an opening of the inlet connection port when the elastomeric valve is in an unbiased state.

18. The airway adapter assembly of claim 14, wherein the inlet connection port of valve connector assembly comprises an elongate channel section extending from an opening of the inlet connection port and having an interior surface for defining at least part of the cavity, the elongate channel comprising an inwardly-extending retaining ridge disposed on the interior surface proximate to the opening.

19. The airway adapter assembly of claim 18, wherein the elastomeric valve comprises a valve face that is countersunk within the elongate channel section relative to the opening of the inlet connection port when the elastomeric valve is in an unbiased state.

20. A method for cleaning a catheter in a suction catheter system, the method comprising:
positioning a tip end of a suction catheter within a cleaning zone of an airway adapter;
coupling a dispenser tip of a solution dispenser to an inlet port of a wash port valve assembly comprising a housing and an elastomeric valve, the wash port valve assembly being controllably fluidly coupled to the cleaning zone of the airway adapter; and
causing solution from the solution dispenser to the flow into cleaning zone.

21. The method of claim 20, further comprising:
decoupling the dispenser tip of the solution dispenser from the inlet port of the wash port valve assembly such that when the dispenser tip is decoupled, the wash port valve assembly is configured to occlude fluid passage from the cleaning zone of the airway adapter to an opening of the inlet port of the wash port valve assembly.
